(19) United States
(12) Patent Application Publication
(10) Pub. No.: US 2011/0066141 A1
(43) Pub. Date: Mar. 17, 2011

(54) IMPLANTABLE MEDICAL DEVICE HAVING AN ANTI-GASTRIC DISTRESS AGENT

(75) Inventors: Amy Lee Hruska, Indianapolis, IN (US); Daniel J. Sirota, Bloomington, IN (US)

(73) Assignee: Cook Incorporated, Bloomington, IN (US)

(21) Appl. No.: 12/557,963

(22) Filed: Sep. 11, 2009

(51) Int. Cl. A61K 9/22

(52) U.S. Cl. 604/890.1

(57) ABSTRACT

Medical devices, and methods of use therewith, suitable for at least partial implantation within the gastrointestinal portions of a body and having at least one anti-gastric distress agent are provided. The anti-gastric distress agent can include antacids, H2 blockers, proton pump inhibitors, or any combination thereof. The medical device can also include one or more sections having one or more anti-gastric distress agent or antimicrobial agents. The medical device has a length such that when the device is at least partially implanted the first section is accessible to the gastrointestinal portion of the patient's body. A second section having an antimicrobial agent may be external to the gastrointestinal portion. Preferably, device includes a first and second anti-gastric distress agent each having a different controlled release rate. The therapeutic agents can be disposed on one or more surfaces of the medical device and/or impregnated with the material of the medical device.

### BACKGROUND

The described embodiments generally relate to medical devices suitable for at least partial implantation into a body. More particularly, they relate to medical devices having at least an anti-gastric distress agent, and methods of use thereof.

Patients for whom normal ingestion of food becomes difficult or impossible may require placement of a feeding tube to assist in providing their nutritional needs. For some individuals, such as comatose patients, stroke victims, or those with a compromised gastrointestinal tract, this may require placement of a feeding tube that is introduced into the stomach for delivery of nutritional products into the stomach. The nutritional products are delivered to the interior of the stomach through the distal end of the feeding tube and/or through side ports along the length of the tube.

Feeding tubes inserted percutaneously through a hole in the abdominal and stomach wall, or gastrostomy, are generally referred to as gastrostomy tubes, or “G”-tubes. In some patients, feeding may be accomplished by inserting a tube, referred to as a jejunostomy tube, or “J”-tube or “GJ”-tube, that extends on through the stomach and directly into the jejunum (the middle section of the small intestine). In many cases, a J-tube may be inserted into the jejunum through the interior of a G-tube that has been previously positioned in the stomach, e.g., via the Seldinger percutaneous entry technique. Feeding tubes may also be inserted into the patient's nose, through the nasal passage, down the esophagus and into the stomach or the small intestines. Collectively, these types of feeding tubes are called nasogastric tubes, or “NG”-tubes. Although its delivery is relatively noninvasive, NG-tubes may contribute to local irritation or infection in the patient's nasal passage.

While indwelling feeding tubes provide several advantages, they also present opportunities for infection. Indwelling feeding tubes are particularly susceptible to infection due to their long term presence in the body. In essence, the feeding tube provides a path from the external environment into the body along which microorganisms can colonize, and eventually produce an infection. Successful colonization along the surface of feeding tubes can even form biofilm layers along its surface. Leakage of gastric contents out of the stomach and into the surrounding skin may also result in inflammation, ulceration, and infection. Gastric contents can include a mixture of nutritional products, dissolved or suspended medications, and gastric secretions. One way to control infection is to clean the feeding tubes periodically by flushing with suitable fluids. Another way is to clean the surrounding skin and to keep it dry.

For patients having feeding tubes and abnormally high leakage of gastric contents, gastroesophageal reflux disease (GERD) or acid reflux, peptic ulcers, gastritis, or excess stomach gas, treatment may be difficult. The oral administration or consumption of antacids to treat excess gastric acid and relieve its associated discomfort is well known.

Although the active ingredients of commercially available, over-the-counter antacid compositions vary, many of these antacids include alkaline earth (e.g., calcium and magnesium) carbonates and hydroxides. More specifically, antacids are generally available in tablet form or liquid form to neutralize acids in the gastrointestinal tract. Antacids are generally insoluble inorganic salts such as calcium hydroxide, aluminum hydroxide, magnesium hydroxide (MILK of MAGNESIA®), combination of aluminum hydroxide and magnesium hydroxide (MMLOX® or MYLANTA®), calcium carbonate (TUMS® or ROLAIDS®), magnesium carbonate, or sodium bicarbonate (ALKA-SELTZER®). Other antacids include bismuth subsalicylate (PEPTO-BISMOL®) and magaldrate with simethicone.

Oral administration or consumption of gastric acid blockers aid to block the release of acid into the patient's gastrointestinal tract is also known. Histamine 2 receptor antagonists (H2 blockers) are compounds that block the H2 receptors on parietal cells in the stomach that stimulate the secretion of acid by the stomach. Gastric acid blockers, which may also include antacids, include for example cimetidine (TAGAMET®), ranitidine (ZANTAC®), or famotidine (PEPCID AC®), just to name a few. Another class of medication used for gastric acid control is proton pump inhibitors. Proton pump inhibitors act to block the molecular pump that produces acid in the patient's gastrointestinal tract. Proton pump inhibitors can include for example omeprazole (PRILOSEC®), dexlansoprazole (KAPIDEX®), or esomeprazole (NEXIUM®), just to name a few.

Instead of periodic oral administration of some form of infection regulation and/or acid regulation or regular cleaning, it would be desirable to provide a medical device in a patient's gastrointestinal system, which is designed to prevent infection and/or to relieve or prevent gastric distress or other effects of gastric acid that cause discomfort to the patient.

### SUMMARY

Medical devices suitable for at least partial implantation into a body, and more particularly, having at least one anti-gastric distress agent for partial or full implantation within the gastrointestinal system are provided. Nonlimiting examples of medical devices can include tubes for delivering nutritional products directly into the gastrointestinal system, such as G-tubes or GI-tubes, GJ-tubes, NG-tubes, etc., tubes for delivering other fluid-like materials such as therapeutic agents or drugs, contrast materials or saline, tubes for delivering specified materials to target sites in other body canals, and/or tubes for drainage of fluid or contents. A portion of the medical device further includes at least one anti-gastric distress agent, such as antacids and acid inhibitors (including H2 blockers and proton pump inhibitors), or any combination thereof. The anti-gastric distress agent can promote relief and/or prevent gastric acid buildup, which causes discomfort to the patient, without the hassles of oral administration of such drugs which for some patients is not possible.

In one embodiment, the medical device can include a body having a distal end, a proximal end, and a lumen extending therebetween. The device can further include a first section proximate the distal end of the main body and having a first therapeutic agent comprising at least one anti-gastric distress agent. The body preferably has a length such that when the device is at least partially implanted the first section is accessible to the gastrointestinal portion of the patient's body.

In another embodiment, the medical device can further include a second section proximate the proximal end and having an antimicrobial agent. At least a portion of the second section preferably is disposed adjacent to body tissue outside the stomach of the patient. For example, the second section can be placed adjacent the nasal passage or throat of a patient for a NG-tube, or adjacent the gastrostomy wall for a G-tube. In other embodiments, the medical device can include a bolster system configured to anchor the medical device to the body. The bolster system preferably has a sleeve sized to fit within the gastronomy and receive the device body. The sleeve, instead of or in addition to the second section, can have a section including the antimicrobial agent. At least a portion of the sleeve section is disposed adjacent the gastronomy wall of the patient.

In another embodiment, the medical device can further include a second anti-gastric distress agent at the first section of the device. The second anti-gastric distress agent preferably has a slower controlled release rate than the first anti-gastric distress agent. In one aspect, the first anti-gastric distress agent is configured to provide immediate relief by absorbing excess gastric acid, while the second anti-gastric distress agent is configured to inhibit the production of gastric acid. The second anti-gastric distress agent can be configured to have a longer release rate or time in order to relieve the adverse effects of gastric acid, such as heartburn.

In some embodiments, the first section of the device comprises a first coating and/or a second coating disposed along a portion of at least one of the exterior surface and luminal surface of the medical device's body. Each of the coatings can include one or more anti-gastric distress agents. Some coatings are biodegradable and are configured to degrade with interaction with the contents of the gastrointestinal tract and/or flushing or feeding. The material of the body may also include and/or be impregnated with one or more anti-gastric distress agents. The device body may further include a second section disposed longitudinally distal to the first section, where the first section of the device includes one or more anti-gastric distress agents and the second section includes one or more anti-gastric distress agents. The cross-sectional area of the second section may be smaller than the first section.

In another aspect, methods of treating gastric distress in a gastrointestinal portion of a body of a patient are provided. One embodiment of the medical device can be introduced into said gastrointestinal portion of the body. The medical device may have any combination of one or more therapeutic agents. The medical device may be introduced through a gastronomy formed in the patient and/or through the nasal passage of the patient. Another step includes at least partially implanting the medical device into the gastrointestinal portion of the body such that the first section is accessible to the gastrointestinal portion of the patient's body.

The above, as well as other advantages of the preferred embodiments, will become readily apparent to those skilled in the art from the following detailed description of a preferred embodiment when considered in the light of the accompanying drawings.

### BRIEF DESCRIPTION OF THE DRAWINGS

FIG. 1 is a perspective view of one embodiment of a medical device.

FIGS. 2A-2D are cross-sectional views taken along line 2-2 in FIG. 1, depicting various embodiments of the medical device.

FIGS. 3A-3D are cross-sectional views taken along line 3-3 in FIG. 1, depicting various embodiments of the medical device.

FIG. 4 is a schematic illustration of a medical device implanted within the gastrointestinal system of a body.

FIG. 5A is a close-up view of a medical device implanted within the gastrointestinal system of the body of FIG. 4.

FIG. 5B is a close-up view of a medical device implanted within the gastrointestinal system of the body of FIG. 4.

FIG. 6 is perspective view of another embodiment of a medical device.

### DESCRIPTION OF PREFERRED EMBODIMENTS

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the invention. The materials, methods, and examples disclosed herein are illustrative only and not intended to be limiting.

In the following discussion, the terms “proximal” and “distal” will be used to describe the opposing axial ends of the inventive device, as well as the axial ends of various component features of the device. The term “proximal” is used in its conventional sense to refer to the end of the device (or component thereof) that is closest to the operator during use of the device. The term “distal” is used in its conventional sense to refer to the end of the device (or component thereof) that is initially inserted into the patient.

The term “medical device” will be used to describe devices for partial or full implantation within the gastrointestinal system, including but not limited to: tubes for delivering nutritional products directly into the gastrointestinal system, or particularly, the stomach and/or the jejunum of a patient, such as G-tubes or GI-tubes, GJ-tubes, NG-tubes, etc; for delivering other fluid-like materials such as drugs, contrast materials or saline; for delivery of specified materials to target sites in other body canals; and/or for drainage of fluid or contents, for example for drainage of the gastric fluid.

The term “anti-gastric distress agent” will be used to describe any or all types of therapeutic agents configured to, at least one of, interact, neutralize, inhibit, reduce, or eliminate gastric hyperacidity or the adverse effects of gastric acid. Specific non-limiting anti-gastric distress agents are described below, including antacids, H2 blockers, and proton pump inhibitors. However, other types of anti-gastric distress agents are known by those skilled in the art are considered within the scope and spirit of the present embodiments.

The term “antimicrobial agent” means any agent that has killing or growth inhibiting effects on one or more microorganisms.

The term “biodegradable material” refers to a material that dissipates upon implantation within a body, independent of the mechanisms by which dissipation can occur, such as dissolution, degradation, absorption and excretion. The actual choice of which type of materials to use may readily be made by one of ordinary skill in the art. Such materials are often referred to by different terms in the art, such as “bioresorbable,” “bioabsorbable,” or “biodegradable,” depending upon the mechanism by which the material dissipates. The prefix “bio” indicates that the erosion occurs under physiological conditions, as opposed to other erosion processes, caused for example, by high temperature, strong acids or bases, UV light or weather conditions.

The term “carrier material” refers to a material that forms a mixture with a therapeutic agent on a surface or in an implantable medical device. The carrier material may control the release of the therapeutic agent from the medical device.

The term “barrier layer” is any layer that is placed over at least a portion of a therapeutic agent present in or on an implantable medical device. In general, the therapeutic agent will not be present in the barrier layer. Any mixing of a therapeutic agent with the barrier layer is unintentional and merely incidental. The barrier layer may or may not be the outer-most layer present on the device. For example, a therapeutic agent may be coated onto a surface of a device, a first barrier layer placed over the therapeutic agent and further barrier layers and layers containing the same or a different therapeutic agent placed on the first barrier layer. The barrier layer may control the, release of the therapeutic agent from the medical device upon implantation.

The term “controlled release” refers to the release of an agent at a predetermined rate. A controlled release may be constant or vary with time. A controlled release may be characterized by a drug elution profile, which shows the measured rate that the agent is removed from a device in a given solvent environment as a function of time. For example, a controlled release elution profile from a medical device may include an initial burst release associated with the implantation of the medical device, followed by a more gradual subsequent release. A controlled release may be a gradient release in which the concentration of the agent released varies over time or a steady state release in which the agent is released in equal amounts over a certain period of time (with or without an initial burst release).

As used herein, the term “therapeutic agent” refers to any pharmaceutically active agent that produces an intended therapeutic effect on the body to treat or prevent conditions or diseases.

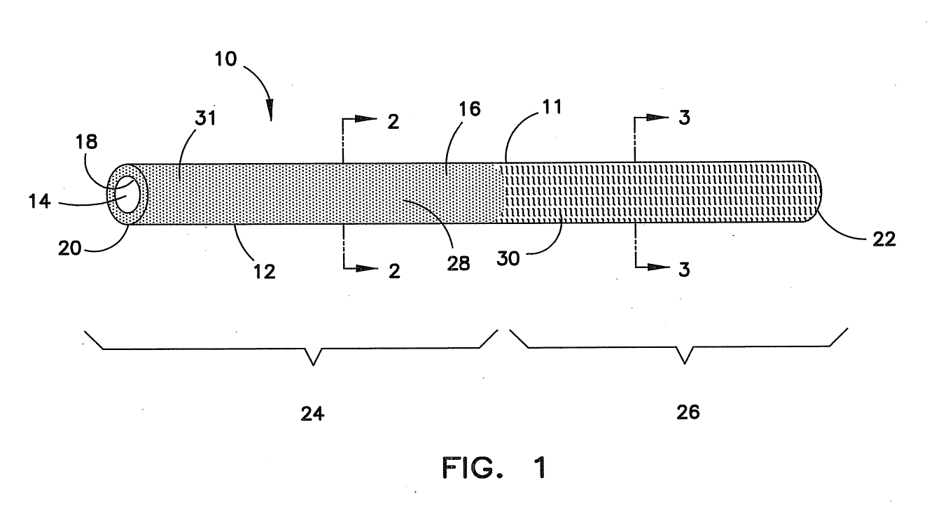

Now referring to all of the figures where like reference numerals are used throughout all of the figures to refer to generally like components, FIG. 1 illustrates one embodiment of a medical device 10 having at least one therapeutic agent for partial or full implantation within the body, preferably within the gastrointestinal system. In this embodiment, the medical device 10 comprises a cannula 11 having a main body 12 defining a lumen 14 about a longitudinal axis. The-medical device 10 includes an exterior surface 16 and an interior surface 18, and a distal end 20 and a proximal end 22. The length of the cannula 11 extends from the distal end 20 to the proximal end 22. A first section 24 of the cannula 11 extends along a portion of the length, and a second section 26 extends along another axially distinct portion of the length. Both the first and second sections 24, 26 extend at least partially circumferentially around the cannula 11 and at least partially axially along a respective portion of the length. As illustrated in FIG. 1, the first section 24 is preferably near the distal end 20 and the second section 26 is preferably near the proximal end 22.

The cross-sectional shape of the medical device can be any shape suitable for the types of procedures in which the device will be utilized. A circular cross-sectional shape is particularly preferable in embodiments in which the device comprises a cannula, such as that illustrated in FIG. 1. A circular cross-sectional shape maximizes space within the lumen 14 of the cannula 11 while also providing a suitable shape for interfacing with a body organ or part. Furthermore, the medical device can have any suitable configuration of lumen(s), and the chosen configuration will depend on the application for which the device is used. Single and multi-lumen configurations can be utilized. U.S. Pat. No. 7,306,580, which is hereby incorporated by reference in its entirety, illustrates various suitable cross-sectional shapes and lumen configurations for use in medical devices according to the present invention.

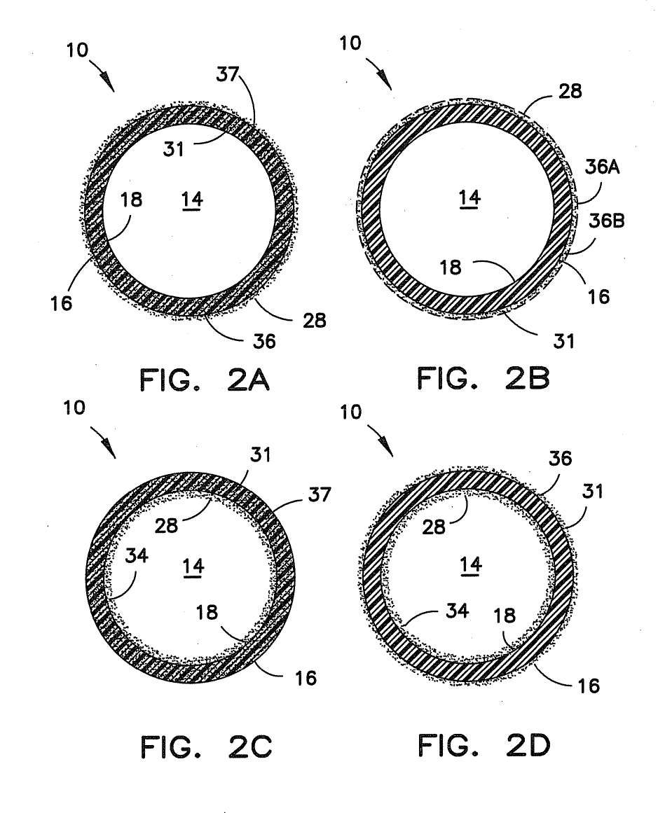

The medical device 10 includes at least one of various therapeutic agents for at least relieving and/or preventing at least one of undesirable gastric acid buildup, preventing infection, or any combinations thereof. For example, in the embodiment shown in FIG. 1, the medical device includes a first therapeutic agent 28 at the first section 24 of the cannula 11, and a second therapeutic agent 30 at the second section 26 of the cannula 11. In other embodiments, the medical device 10 can include more therapeutic agents, for example, a third therapeutic agent 31 as shown in FIGS. 2B-2D.

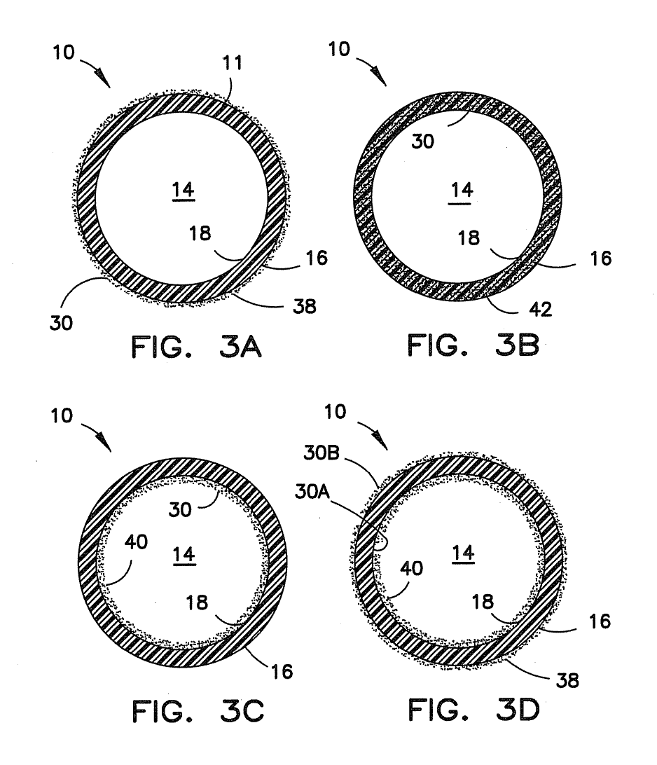

The therapeutic agents 28, 30, 31 can be associated with the cannula 11 in a variety of manners described below. Any suitable technique for placing a therapeutic agent in, on, or near a medical device for release from the medical device can be utilized, For example, in the cross-sectional view illustrated in FIG. 2A, the external surface 16 of the first section 24 of the cannula 11 includes a coating layer 36 comprising a first therapeutic agent 28 posited thereon. A portion 37 of the material of the first section 24 can include the same or another therapeutic agent 31 impregnated within the material. Also, in the cross-sectional view of a preferred embodiment illustrated in FIG. 3A, the external surface 16 of the second section 26 of the cannula 11 includes a coating layer 38 comprising a second therapeutic agent 30 posited thereon. Other embodiments are described in more detail below.

Many alternative embodiments of the orientation and placement of the therapeutic agents are within the scope of the present invention. For example, FIGS. 2B-2D illustrate cross-sectional views of alternative embodiments of the first section 24 of the cannula 11, and FIGS. 3B-3D illustrate cross-sectional views of alternative embodiments of the second section 26 of the cannula 11.

In FIG. 2B, a first coating layer 36A comprises a first therapeutic agent 28, represented by the dashed line, and a second, more inner coating layer 36B comprises another therapeutic agent 31, each disposed on the exterior surface 16 of the cannula 11. In FIG. 2C, a coating layer 34 disposed on the interior surface 18 of the cannula 11 comprises a first therapeutic agent 28 and a portion 37 of the material of the first section 24 comprises another therapeutic agent 31 impregnated therein. In FIG. 2D, the coating layer 34 disposed on the interior surface 18 of the cannula 11 comprises a first therapeutic agent 28, and the coating layer 36 disposed on the exterior surface 16 of the cannula 11 comprises another therapeutic agent 31. In other embodiments, multiple coating layers and impregnated materials can each include a single therapeutic agent. In other embodiments, a coating layer or impregnated material can include a combination of therapeutic agents.

Similarly, the second therapeutic agent 30 can be associated with the second section 26 of the cannula 11. For example, in FIG. 3B, a portion 42 of the material of the second section 26 of the cannula 11 comprises a second therapeutic agent 30 impregnated therein. In FIG. 3C, a coating layer 40 disposed along the interior surface 18 of the cannula 11 comprises a second therapeutic agent 30. In FIG. 3D, one or more therapeutic agents 30A, 30B can be included in two coatings: a coating layer 38 disposed on the exterior surface 16 and a coating layer 40 disposed on the interior surface 18. In addition to the various arrangements for each of the therapeutic agents, any suitable combination of arrangements may be utilized and/or more than one first, second, third therapeutic agents may be utilized with the medical device. For example, although some of the figures illustrate more than one coating, some embodiment may include only one coating with one therapeutic agent.

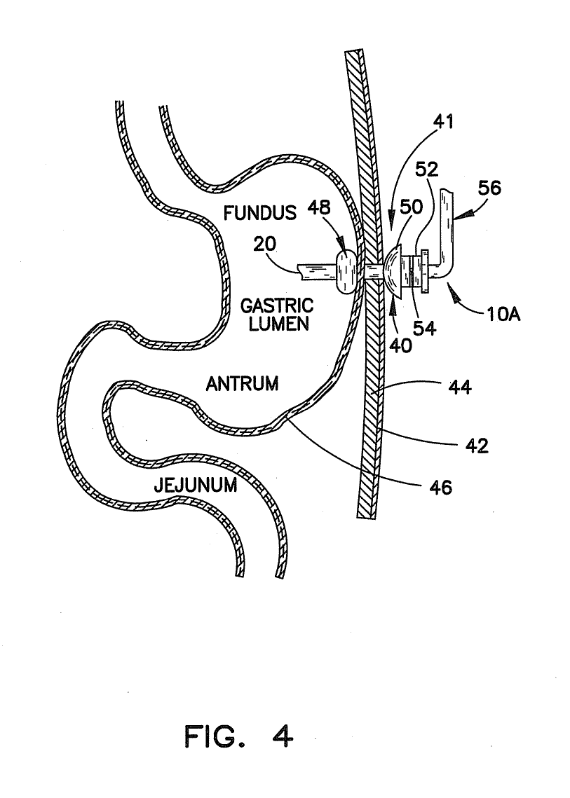

FIG. 4 schematically illustrates the relative position of at least a partially implanted embodiment of the medical device 10A and a bolster system 41 when the device is inserted through an opening in the abdominal wall of a patient and into the gastrointestinal system, or more specifically the stomach. The bolster system 41 can include an external bolster member 40 and an internal bolster member 48. The external bolster member 40 is in cooperating relationship with the medical device 10A of the type that extends from the exterior of the patient to a site internal of the patient. In the non-limiting example illustrated herein, the medical device 10A is an enteral feeding tube, such as a gastrostomy tube, or G-tube. As illustrated, the distal end 20 of the medical device 10A extends through an epidermal surface 42 and other layers of the skin, through subcutaneous layers including an abdominal wall 44 and a stomach wall or lining 46, and into the stomach. Some main components of the gastrointestinal system are also depicted in the figure including: the fundus of the stomach; the main body, or gastric lumen, of the stomach; the antrum of the stomach which is the portion before the outlet; and the jejunum which is the middle section of the small intestines.

FIG. 4 illustrates one embodiment of the external bolster 40. In the embodiment shown, the external bolster 40 is provided external of the patient for anchoring the medical device 10A along the patient's skin. The external bolster 40 includes a base portion 50 that is positioned in a manner to abut, or substantially abut, the external epidermal surface 42, and an elongated body portion 52 that is configured to receive and surround the medical device 10A. An optional securing structure, such as twist lock member 54, may be provided around the elongated body 52 for applying a gripping pressure on the medical device 10A. Preferably, the base portion 50 comprises the dome-like configuration. Utilizing a dome-like configuration at the distal portion of the base for contacting the skin of the patient provides bolstering with a limited ability to rock back and forth upon movement of the medical device 10A. Excessive rocking movement can cause trauma to the skin of the patient.

The internal bolster member 48 depicted is one example of the type of internal bolster used with tubes. Typically, the internal bolster 48, such as a balloon or a flared portion of the tube, is provided around a portion of the medical device 10A. The internal bolster is positioned internal of the stomach to abut and hold the anterior stomach wall 46 against the abdominal wall 44. The internal and external bolsters 48, 40 are configured to maintain the surface 42 of the body and the internal surface of the stomach wall 46 in close proximity. After coupling, the external bolster 40 is secured to the medical device 10A in order to prevent future migration of the device and reduce the need for constant reposition or pull on the device. In the preferred embodiments disclosed, the bolster system may be formed separately or as an integral unit, and the segments of the system may be formed of the same, or a similar, composition. One bolster system is described in U.S. Patent Publ. No. 2008/0082081 A1, which is hereby incorporated by reference in its entirety. Other types of bolster systems are described in U.S. Pat. Nos. 5,716,347 and 5,720,734, which are hereby incorporated by reference in their entirety. It is to be understood by one of ordinary skill in the art that, instead of using the described bolster system, the proximal end of the medical device 10A can reside, and otherwise be fixed, in other locations such as the patient's nose, and similarly the distal end can reside in other locations such as the jejunum.

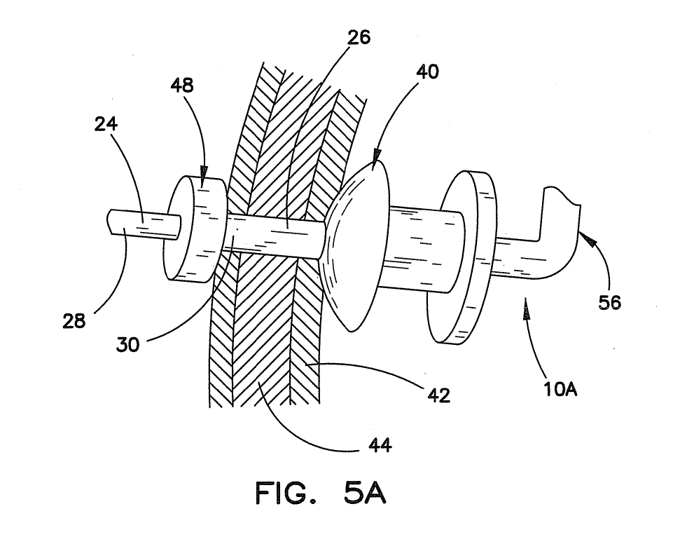

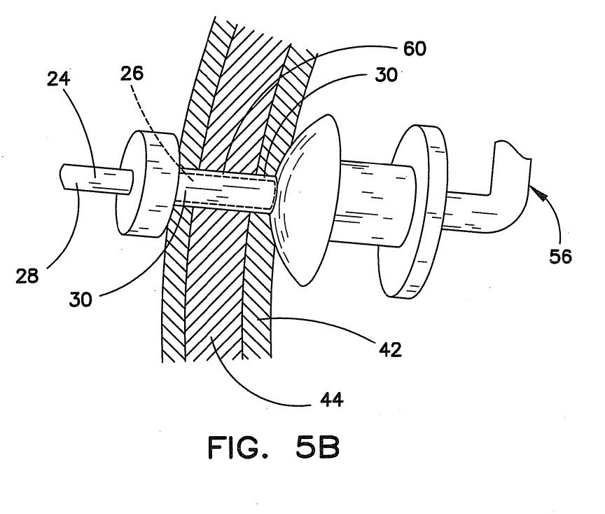

FIG. 5A is a close-up view of another embodiment of the bolsters 40, 48 and the medical device 10A partially implanted within the body such that the lumen of the medical device 10A is in communication with the gastrointestinal system. In particular, at least a portion of the second section 26 of the feeding tube is in direct interface with subcutaneous layers such as at least one of the epidermal surface 42, the abdominal wall 44 and the stomach wall 46. A portion 56 of the device 10A remains external to the body. This external portion 56 provides the desired access to the lumen which is in communication with the gastrointestinal system. Thus, the gastrointestinal system can be accessed without further disruption to the skin. In this example, a portion of the second section 26 includes one or more second therapeutic agents 30 and is preferably positioned across the subcutaneous layers. The second section 26 may even extend below the stomach wall. Because of this position of the second section 26, the second therapeutic agent 30 can inhibit infection of the contacting portions of the medical device 10A with the body. A portion of the first section 24 includes the first therapeutic agent 28, which is positioned within the gastrointestinal system. Because of the position of the first section 24, the first therapeutic agent 30 can inhibit gastric acid buildup or adverse effects thereof.

FIG. 5B is a close-up view of another embodiment of the bolsters 40, 48 and the medical device 10A partially implanted within the body as described above. FIG. 5B, however, illustrates a sleeve 60 or barrier coupled to at least one of the bolsters 40, 48 such that the sleeve 60 is in direct interface with the subcutaneous layers. The sleeve 60 which has a lumen configured to surround and receive the medical device 10A can be made of materials similar to the aforementioned materials of the cannula. In this example, a portion of the sleeve 60, rather than the second section 26, includes one or more second therapeutic agents 30, and is preferably positioned across at least one of the epidermal surface 42, the abdominal wall 44 and the stomach wall 46. Because of this position of the sleeve 60, the second therapeutic agent 30 can inhibit infection of the contacting portions of the medical device and/or sleeve with the body. The second section 26 of the medical device preferably does not have any therapeutic agent. A portion of the first section 24 includes the first therapeutic agent 28, which is positioned within the gastrointestinal system for inhibiting gastric acid buildup or adverse effects thereof. It can be appreciated that the components of the bolster system may also include therapeutic agents for respectively inhibiting infection or gastric acid buildup.

For NG tubes, the second section 26 having one or more second therapeutic agents 30 can be associated along various portions of the nasal passage, along various portions of the throat, and/or along various portions of the esophagus. In this instance, the second therapeutic agent is preferably positioned across the respective portions in order for the second therapeutic agent to inhibit infection of the contacting portions of the medical device with the body. For example, the second section can extend from the proximal end of the device for a longitudinal length sufficient to contact portions of the nasal passage and the throat. In another example, the second section may have a first region for contacting a portion of the nasal passage and a second region for contacting a portion the throat, with each of the first and second regions having the same second therapeutic agent or different second therapeutic agents for treating each respective area.

The therapeutic agents can be any suitable agents, and need only provide the desired effects. Thus, the therapeutic agents which are associated with the first section 24 can include anti-gastric distress agents that can have a negative effect on the formation or the neutralizing effect on gastric acids. Also, the therapeutic agent which is associated with the second section 26 can have an antimicrobial effect.

The therapeutic agents 28, 31 associated with the first section 24 can be any suitable anti-gastric distress agent. For example, the therapeutic agents 28, 31 can comprise one or more antacids including a variety of alkaline substances. Examples include but not limited to any antacids acceptable to the Food and Drug Administration, such as aluminum carbonate, aluminum hydroxide (or as aluminum hydroxide-hexitol stabilized polymer, aluminum hydroxide-magnesium hydroxide codried gel, aluminum hydroxide-magnesium trisilicate codried gel, aluminum hydroxide-sucrose powder hydrated), aluminum phosphate, aluminum hydroxy carbonate, dihydroxy aluminum sodium carbonate, aluminum magnesium glycinate, dihydroxy aluminum aminoacetate, dihydroxyaluminum aminoacetic acid, bismuth aluminate, bismuth carbonate, bismuth subcarbonate, bismuth subgallate, bismuth subnitrate, bismuth subsalicylate, calcium carbonate, calcium phosphate, calcium hydroxide, hydrated magnesium aluminate activated sulfate, magnesium aluminate, magnesium aluminosilicates, magnesium carbonate, magnesium glycinate, magnesium hydroxide, magnesium oxide, magnesium trisilicate, sodium bicarbonate, potassium bicarbonate, glycine, dried milk solids, sodium carbonate, potassium carbonate, sodium potassium tartrate, and magaldrate with simethicone. A combination of antacids may also be used. Preferably, the antacid is selected from aluminum hydroxide, calcium carbonate, magnesium carbonate, and magnesium hydroxide. Other antacids are known to those skilled in the art and can be found in standard reference literature such as Remington, the Science and Practice of Pharmacy 20th Ed. and the United States Pharmacopeia (USP 26).

Antacids can treat an abnormally high acidic gastric acid (pH of about 2) by raising the pH level of the stomach to between 3 and 4. This can neutralize up to 99 percent of the gastric acid, leading to substantial relief from symptoms for most people. Antacids can work within a minute, and can provide relief that ranges from 10 minutes to more than 90 minutes. Generally, antacid compositions containing weaker acid neutralizing agents such as calcium carbonate and aluminum hydroxide are slower acting and consequently do not provide as rapid relief to the discomfort associated with excess or highly acidic gastric acid, but may last longer. More rapid acting antacids may include a stronger acid neutralizing agent, such as, for example, magnesium hydroxide or sodium bicarbonate, but may not last as long.

Alternatively, or in combination with the antacids, the therapeutic agent 28, 31 can include one or more gastric acid blockers including a variety of H2 blockers. Examples of H2 blockers include cimetidine, famotidine, nizatidine, ranitidine, and ranitidine bismuth citrate. H2 blockers are known to provide longer lasting relief than antacids, up to about 6 to 10 hours or more, depending on the dosage.

Alternatively, or in combination with the antacids and/or H2 blockers, the therapeutic agents 28, 31 can include one or more proton pump inhibitors. The proton pump inhibitor may be, if desired, in the form of free base, free acid, salt, ester, hydrate, anhydrate, amide, enantiomer, isomer, tautomer, prodrug, polymorph, derivative, or the like, provided that the free base, salt, ester, hydrate, amide, enantiomer, isomer, tautomer, prodrug, or any other pharmacologically suitable derivative is therapeutically active. Examples included omeprazole, dexlansoprazole, esomeprazole, lansoprazole, pantoprazole, pariprazole, leminoprazole, rabeprazole, tenatoprazole, and derivatives thereof. Proton pump inhibitors can provide longer lasting relief than antacids and H2 blockers, for example, up to about 2 to 3 days or more, depending on the dosage. Proton pump inhibitors can be prescribed for treatment of active duodenal ulcers, gastrointestinal ulcers, gastro esophageal reflux disease (GERD), severe erosive esophagitis, poorly responsive symptomatic GERD, and pathological hypersecretory conditions such as Zollinger Ellison syndrome.

The therapeutic agent 30 which is associated with the second section 26 can be any suitable antimicrobial agent. Suitable classes of antimicrobial agents include antibiotics, disinfectants, and antiseptics.

In a preferred embodiment, the therapeutic agent 30 comprises one or more antibiotics having activity against the common microorganisms associated with colonization and/or infection with indwelling cannula. Examples of suitable classes of antibiotics include tetracyclines, rifamycins, macrolides, penicillins, cephalosporins, other beta-lactam antibiotics, aminoglycosides, chloramphenicol, sulfonamides, glycopeptides, quinolones, fusidic acid, trimethoprim, metronidazole, clindamycin, mupirocin, polyenes, azoles and beta-lactam inhibitors.

Examples of specific antibiotics that can be used as the therapeutic agent 30 include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin.

The therapeutic agent 30 can comprise a combination of two or more antimicrobial agents. In these embodiments, the two or more antimicrobial agents can be located in or on discrete locations within the second section 26, or the two or more antimicrobial agents can be blended together and uniformly distributed within or on the second section 26. In a preferred embodiment, rifampin and minocycline are used as the therapeutic agent 30. The rifampin and minocycline preferably are blended together and evenly distributed either in or on the second section 26. In a particularly preferred embodiment, discussed below, blended rifampin and minocycline are coated onto the surfaces of second section 26. With reference to FIG. 3D, it may be advantageous for one embodiment of the medical device to have a first antimicrobial agent 30A associated with the interior surface 18 and configured to reduce infection caused by contents entering into, or exiting from, the gastrointestinal system. Further, the embodiment of the medical device includes a second antimicrobial agent 30B, different than the first, associated with the exterior surface 16 and configured to reduce infection caused by the outside environment or the wall of the gastrostomy.

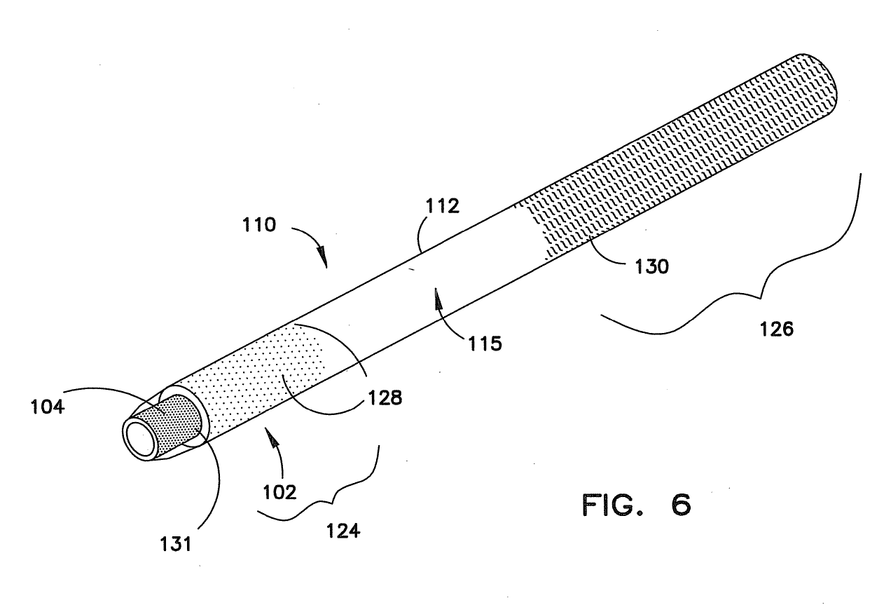

FIG. 6 illustrates one aspect of the medical device 110 that can be included with any of the embodiments described herein, which includes a separator 115 configured to space the first section 124 from the second section 126. The separator 115, in addition to physically separating the first and second sections 124,126, provides a visual indicator of the transition between these sections, which can aid fabrication and implantation procedures. The separator 115 can be any suitable separator that provides a separation between the first and second sections 124,126. The separator 115 need only not interfere with implantation in the body. In some embodiments, the separator 115 comprises a portion of the main body 112 that has a reduced diameter as compared to the diameters of the first and second sections 124,126. Examples of other suitable separators include markers, such as bands and dyes disposed within or on the main body and other visual indicators.

FIG. 6 also illustrates another aspect of the medical device 110 that can be included with any of the embodiments described herein, which includes a first elongated member 102 and a discrete second elongated member 104 (or a reduced diameter portion of the first tube). The first elongated member is sized to fit within a larger cross-sectional area, while the second elongated member 104 is sized to fit within a smaller cross-sectional area, such as for example the jejunum.

Another aspect in FIG. 6 is the application of therapeutic agents in a side-by-side configuration. For example, the first section 124 of the first elongated member 102 can be configured to interact with the stomach. To this end, the first section 124 can include a therapeutic agent 128 comprising at least one anti-gastric distress agent that is configured to interact more effectively within the stomach. The second elongated member 104 includes another therapeutic agent 131 comprising at least one anti-gastric distress agent that is configured to interact more effectively within the intestines. The second section 126 can also include another therapeutic agent 130 comprising an antimicrobial agent, as described above. Various non-limiting configurations for applications of the therapeutic agents are described above with reference to FIGS. 2A-2D and FIGS. 3A-3D. It is to be understood that the side-by-side configuration can be included in various embodiments of medical devices 10 or 10A, as well as for the second sections of the various embodiments of the medical devices.

The cannula of the medical device, or various sections or elongated members of the cannula, can be made from any suitable material(s) acceptable for use in a medical device and acceptable for the intended use of the medical device. Preferably, the material(s) is able to have one or more therapeutic agents associated with it. Examples of suitable material(s) include polymers, copolymers, plastics, and metals. Typically, the material(s) is a biocompatible polymeric material, including but not limited to silicone, polyurethane, a copolymer of silicone and polyurethane, or a polyamide, such as nylon. The material(s) chosen will depend on several factors, including the intended use of the medical device, the therapeutic agents that will be used in the medical device, the ability of the material to have one or more of the agents associated with it, the permeability of the material to the therapeutic agents, and the ability of the material to be formed into members permeable to the therapeutic agents. The cannula may have portions of different materials, for example the first and second elongated members of FIG. 6 can be formed of different materials beneficial for their respective implantation.

The therapeutic agent(s) can be associated with the respective portions of the medical device in any suitable manner. For example, if a therapeutic agent(s) is bulk distributed in the material of the medical device, a swelling method can be utilized. Alternatively, the therapeutic agent(s) can be added to a melt of bulk material. Once extruded, the medical device will include the therapeutic agent(s) in the material. Further, if different portions of the medical device have therapeutic agents associated in different manners (e.g., bulk distribution versus coating layer), a combination of suitable techniques can be utilized. The separator between the first and second sections of medical devices according to these embodiments, as described above, can advantageously be used to isolate different techniques during fabrication.

The therapeutic agent(s) can be releasably associated with the medical device, meaning that the therapeutic agent can be released from the medical device upon implantation in the body of a patient. Preferably, the therapeutic agent(s) is released in a controlled manner. The therapeutic agent(s) can be included in any suitable part or component of the medical device cannula or other components used in association with the medical device. As described in various embodiments, the therapeutic agent(s) can be incorporated within the material of and/or coated onto the surface of one or more of these elements. The therapeutic agent(s) can also be placed in holes, wells or groves formed in the cannula of the medical device.

A porous barrier layer can be posited over some, or all, of the therapeutic agent(s) to control the release of the therapeutic agent(s) from the medical device. Multiple porous barrier layers and/or the pore size in the porous barrier layer can be used to control the rate of release of the therapeutic agent(s). One or more therapeutic agents may be impregnated into the material of the medical device or components used therewith. Methods of impregnating therapeutic agents into the structure of non-metallic medical devices are described in U.S. Pat. No. 5,624,704, which is hereby incorporated by reference. One or more therapeutic agents may be impregnated into such medical devices by contacting the medical device with the therapeutic agent in a suitable solvent. In some cases, a penetrating ingredient is also added.

In preferred embodiments, one, first anti-gastric distress agent has a faster controlled release rate than another, second anti-gastric distress agent. In other embodiments, one anti-gastric distress agent has a slower treatment rate than another anti-gastric distress agent. Accordingly, the first anti-gastric distress agent is configured to interact with the gastric acid or the gastric acid source to provide quicker relief, while the second anti-gastric distress agent is configured to interact with the gastric acid and/or gastric acid source in order to provide relief over extended periods of time. Examples of combinations may include the first anti-gastric distress agent including a faster acting antacid such as magnesium hydroxide or sodium bicarbonate and the second anti-gastric distress agent including a slower acting antacid such as calcium carbonate or aluminum hydroxide. Alternatively, the first anti-gastric distress agent can include an antacid and the second anti-gastric distress agent can include a H2 blocker and/or a proton pump inhibitor.

Selection of the type of anti-gastric distress agent(s), the portions of the medical device containing the anti-gastric distress agent(s) and the manner of attaching the anti-gastric distress agent(s) to the medical device can be chosen to perform a desired therapeutic function upon implantation and, in particular, to achieve the controlled release of the anti-gastric distress agent(s) at a desired rate.

Also, if a coating layer is desired, the therapeutic agent(s) can be dip-coated, spray-coated, or coated onto the medical device using any other suitable coating technique. The therapeutic agent(s) can be coated directly on a medical device surface as a separate layer. The therapeutic agent(s) can be bonded to the surface directly via a covalent bond or via a linker molecule which covalently links the therapeutic agent(s) and the surface. The therapeutic agent(s) can also be bound to the surface by ionic, hydrophobic or hydrogen bonding interactions.

Alternatively, the therapeutic agent(s) can be attached to a medical device surface within a layer including a carrier material. For example, the therapeutic agent(s) can be mixed with the carrier material, such as a polymer, and applied to a surface of the medical device, for example, by spray or dip coating onto the surface. If the carrier material is biostable, the therapeutic agent(s) can be released by diffusion through the carrier material. If the carrier material is biodegradable, the therapeutic agent(s) can be released upon erosion of the biodegradable carrier material.

The carrier material may include a biostable polymer, a biodegradable polymer or any combination thereof. In one embodiment, the therapeutic agent(s) is blended with a biostable polymer to deposit the therapeutic agent(s) within the porous channels within the biostable polymer that permits release of the therapeutic agent(s) from the medical device upon implantation. Alternatively, a blend of the therapeutic agent(s) and a biodegradable polymer can be incorporated within a biostable polymer matrix to permit dissolution of the biodegradable polymer through channels or pores in the biostable polymer matrix upon implantation in the body, accompanied by release of the therapeutic agent(s). In another embodiment, the carrier material includes a biostable polymer, a biodegradable polymer or any combination thereof that is configured to degrade over a certain amount of time such that suitable dosages of therapeutic agent(s) are released periodically to provide relief over a longer implanted time. For example, suitable dosages of therapeutic agent(s) can be released every hour (for example, 0.5%, 1%, 2% or similar increments), every day (5%, 10%, 15% or similar increments) or portions thereof. The percentages may vary due to the type of therapeutic agent, concentration of the therapeutic agent, different expected indwelling times for the medical device, severity of patient's condition, region of gastrointestinal tract, and size of patient.

In another embodiment, for example as illustrated in FIGS. 2C and 2D, the interior coating 34 of the medical device can be more protected from gastric juices of the gastrointestinal tract than an exterior coating, which may extend the life of the associated therapeutic agent. Further, the interior coating 34 can be configured to interact with the fluid used for flushing the medical device and/or the nutrient fluid used for feeding the patient. The coating may have additional carrier and/or barrier layers that degrade when there is flushing or feeding so that the anti-gastric distress agent is released. The interior coating can include anti-gastric distress agents, and more preferably, an anti-gastric distress agent such as antacid that can be released during flushing or feeding. This is particularly useful since patients may be more apt to require faster acting anti-gastric distress agents during feeding than during other times. In addition, as opposed to interacting with gastric juices of the gastrointestinal tract which may cause faster degradation of the coating, interacting with flushing fluid or feeding fluid may cause a more controlled degradation along the interior coating. In other examples, the exterior of the medical device can allow for a more constant release of an anti-gastric distress agent, while there can be additional intermittent treatment when the medical device is flushed or used for feeding.

In accordance with the provisions of the patent statutes, the present invention has been described in what is considered to represent its preferred embodiment. However, it should be noted that the invention can be practiced otherwise than as specifically illustrated and described. Those skilled in the art will recognize that variations and modifications can be made without departing from the true scope and spirit of the preferred embodiments as defined by the claims that follow. It is therefore intended to include within the preferred embodiments all such variations and modifications as fall within the scope of the appended claims and equivalents thereof.

1. A method of treating gastric distress in a gastrointestinal portion of a body, comprising:
introducing a medical device into said gastrointestinal portion of the body, the medical device including a body having a proximal end, a distal end, and a lumen extending therebetween, the body having a first section proximate the distal end thereof, the first section having a therapeutic agent configured to inhibit gastric distress; and
at least partially implanting said medical device into said gastrointestinal portion of the body such that said first section is accessible to said gastrointestinal portion of the body.

2. The method of claim 1, wherein the first section comprises a coating disposed along a portion of an exterior surface of the medical device body, the coating comprising the therapeutic agent.

3. The method of claim 1, wherein the medical device body comprises a material having a portion thereof including the therapeutic agent.

4. The method of claim 1, wherein the first section comprises a coating disposed along a portion of a luminal surface of the medical device body, the coating comprising the therapeutic agent.

5. The method of claim 4, wherein a portion of the therapeutic agent is also configured to controllably release during flushing of the device or feeding of a patient.

6. The method of claim 1, wherein the therapeutic agent is a first anti-gastric distress agent, the first section further comprising a second anti-gastric distress agent, where each of the first and second anti-gastric distress agents comprises at least one of an antacid and an acid inhibitor.

7. The method of claim 6, wherein the first anti-gastric distress agent has a controlled release rate and the second anti-gastric distress agent has a controlled release rate slower than the controlled release rate of the first anti-gastric distress agent.

8. The method of claim 6, wherein the first section comprises a coating disposed along a portion of an exterior surface of the medical device body, the coating comprising the first anti-gastric distress agent, and the medical device body comprising a material having a portion thereof including the second anti-gastric distress agent.

9. The method of claim 6, wherein the first section comprises a coating disposed along a portion of a luminal surface of the medical device body, the coating comprising the first anti-gastric distress agent, and the medical device body comprising a material having a portion thereof including the second anti-gastric distress agent.

10. The method of claim 6, wherein the first section comprises a first coating disposed along a portion of an external surface of the medical device body, and a second coating disposed along a portion of a luminal surface of the medical device body, the first coating comprising one of the first and the second anti-gastric distress agents, the second coating comprising the other of the first and the second anti-gastric distress agents.

11. The method of claim 6, wherein the device body further comprises a second section disposed longitudinally distal to the first section, the first section of the device comprising the first anti-gastric distress agent and the second section comprising the second anti-gastric distress agent.

12. The method of claim 11, wherein the second section has a smaller cross-sectional area than the first section.

13. The method of claim 1, wherein the first section is proximate the distal end of the medical device body, the body further comprising a second section proximate the proximal end of said main body, wherein the therapeutic agent comprises at least one of an antacid and an acid inhibitor, and the second section comprises an antimicrobial agent.

14. The method of claim 13, wherein the second section is separated from the first section by a longitudinal distance.

15. The method of claim 13, wherein the second section is configured to be placed external to the gastrointestinal region of the body.

16. The method of claim 13 further comprising forming a gastrostomy through an epidermal surface and subcutaneous layers of the body to form a wall of the gastrostomy, wherein said medical device is at least partially implanted such that at least a portion of the second section is disposed adjacent the gastrostomy wall of the patient.

17. The method of claim 1 further comprising forming a gastrostomy through an epidermal surface and subcutaneous layers of the body to form a wall of the gastrostomy, wherein the medical device further comprises a bolster system configured to anchor the medical device to the body, the bolster system having a sleeve sized to fit within the gastrostomy and to receive the main body of said device, the sleeve having a section comprising an antimicrobial agent, wherein said medical device is at least partially implanted where at least a portion of the sleeve section is disposed adjacent the gastrostomy wall of the patient.

18. A medical device for at least partial implantation into a gastrointestinal portion of a body, comprising:
a body having a proximal end, a distal end, and a lumen extending therebetween, the body including a first section proximate the distal end thereof, the first section having an anti-gastric distress agent,
said body having a predetermined length such that, when said medical device is at least partially implanted into the body, said first section is accessible to said gastrointestinal portion of the body.

19. The device of claim 18, wherein the body further comprises a second section proximate the proximal end, the second section having an antimicrobial agent, where at least a portion of the second section is configured to be placed external to the gastrointestinal portion of the body.

20. The device of claim 19, wherein the anti-gastric distress agent is a first anti-gastric distress agent, the first section further comprising a second anti-gastric distress agent, wherein each of the first and the second anti-gastric distress agents has a controlled release rate, the controlled release rate of the second anti-gastric distress agent being slower than the first anti-gastric distress agent.
